(19) United States
(12) Patent Application Publication
(10) Pub. No.: US 2024/0238602 A1
(43) Pub. Date: Jul. 18, 2024

(54) IMPLANTABLE MEDICAL DEVICE

(71) Applicant: Shiratronics, Inc., Brooklyn Park, MN (US)

(72) Inventor: Scott A. Spadgenske, Buffalo, MN (US)

(21) Appl. No.: 18/410,594

(22) Filed: Jan. 11, 2024

Related U.S. Application Data
(60) Provisional application No. 63/439,330, filed on Jan. 17, 2023.

(51) Int. Cl. A61N 1/375

(52) U.S. Cl. CPC A61N 1/3752; A61N 1/3758

(57) ABSTRACT

A device may include a housing, at least one lead, stimulator circuitry and a battery. The housing may include a lead retention feature. Each of the lead(s) may include a plurality of electrodes. The stimulator circuitry may include a waveform generator configured to generate an electrical waveform and a controller configured to electrically drive the electrical waveform through at least one of the plurality of electrodes. The battery may be configured to provide electrical power to the stimulator circuitry. The stimulator circuitry and the battery may be within the housing. Each of the lead(s) may include a plurality of electrical conductors extending from a proximal lead end to provide electrical connections to the electrodes. The electrical conductors may be electrically connected to the stimulator circuitry. The proximal lead end may be retained within the housing by the lead retention feature.

### PRIORITY

This application claims the benefit of priority to U.S. Provisional Patent Application Ser. No. 63/439,330, filed Jan. 17, 2023, which is incorporated by reference herein in its entirety.

### TECHNICAL FIELD

This document relates generally to medical systems, and more particularly, but not by way of limitation, implantable medical device systems.

### BACKGROUND

Medical devices may sense electrical signals and/or deliver an electrical therapy. For example, medical devices may include implantable devices configured to deliver a therapy such as an electrical therapy. Implantable electrical therapy devices may include implantable neurostimulators. An example of an implantable neurostimulator includes a fully head-located implantable peripheral neurostimulation system, having one or more implantable devices, designed for the treatment of chronic head pain is a specific example of an implantable neurostimulation system.

### SUMMARY

An example (e.g., “Example 1”) of a device may include a housing, at least one lead, stimulator circuitry and a battery. The housing may include a lead retention feature. Each of the lead(s) may include a plurality of electrodes. The stimulator circuitry may include a waveform generator configured to generate an electrical waveform and a controller configured to electrically drive the electrical waveform through at least one of the plurality of electrodes. The battery may be configured to provide electrical power to the stimulator circuitry. The stimulator circuitry and the battery may be within the housing. Each of the lead(s) may include a plurality of electrical conductors extending from a proximal lead end to provide electrical connections to the electrodes. The electrical conductors may be electrically connected to the stimulator circuitry. The proximal lead end may be retained within the housing by the lead retention feature.

In Example 2, the subject matter of Example 1 may optionally be configured to further include a battery liner adjacent to at least a portion of a periphery of the battery. The battery liner may include at least one spring configured to press against the periphery of the battery to resist battery movement.

In Example 3, the subject matter of Example 2 may optionally be configured such that the battery liner is configured to be positioned along at least a width edge of the battery and a length edge of the battery. The spring(s) may include one or more springs along the width edge of the battery to constrain battery movement in the length direction and one or more springs along the length edge of the battery to constrain the battery movement in the width direction.

In Example 4, the subject matter of any one or more of Examples 2-3 may optionally be configured such that the spring(s) includes an arch spring.

In Example 5, the subject matter of any one or more of Examples 1-4 may optionally be configured such that the device includes a bottom surface corresponding to a contour of a skull.

In Example 6, the subject matter of any one or more of Examples 1-5 may optionally be configured such that the housing of the device includes a base mold, a coil frame and a can assembly. The base mold may have a first portion with a first planar bottom surface and a second portion with a second planar bottom surface. The first and second planar bottom surfaces may form an angle corresponding to a skull contour such that the bottom surfaces for both the first and second portions are approximately tangent to a skull surface. The coil frame may be configured for holding a coil on the first portion of the base mold. The can assembly may be configured for housing the stimulator circuitry on the second portion of the base mold.

In Example 7, the subject matter of Example 6 may optionally be configured such that the coil frame has an interface with the can assembly. Each of the coil frame near the interface and the can assembly near the interface may have features for enhancing an ability of epoxy to fix the coil frame to the can assembly. The epoxy may be provided over the interface and hardens to fix the coil frame to the can assembly.

In Example 8, the subject matter of Example 7 may optionally be configured such that the features for enhancing the ability of the epoxy to fix the coil frame to the can assembly may include apertures in each of the coil frame and the can assembly. The epoxy at least partially fills the apertures.

In Example 9, the subject matter of any one or more of Examples 6-8 may optionally be configured to further include a flexible tail. The flexible tail and the coil frame may be on opposing sides of the can assembly. The base mold may have a third portion with a third planar bottom portion. The flexible tail may be formed on the third portion. The third portion may have a bottom surface forming an angle with the bottom surface of the second portion such that the bottom surface of the third portion is approximately tangent to the skull surface.

An example (e.g., “Example 10”) of a device may include a housing, at least one lead, stimulator circuitry, a battery, a coil, and an epoxy. The housing may include a coil frame and a can assembly. Each of the lead(s) may include a plurality of electrodes. The battery may be configured to provide electrical power to the stimulator circuitry. The stimulator circuitry may include a waveform generator configured to generate an electrical waveform and a controller configured to electrically drive the electrical waveform through at least one of the plurality of electrodes. The stimulator circuitry and the battery may be within the can assembly. The coil may be electrically connected to the stimulator circuitry. The coil frame may be configured to hold the coil. The epoxy may be over an interface between the coil frame and the can assembly to fix the coil frame to the can assembly. Each of the coil frame near the interface and the can assembly near the interface may have features for enhancing an ability of epoxy to fix the coil frame to the can assembly when hardened.

In Example 11, the subject matter of Example 10 may optionally be configured such that the features for enhancing the ability of the epoxy to fix the coil frame to the can assembly includes apertures in each of the coil frame and the can assembly. The epoxy may at least partially fill the apertures.

In Example 12, the subject matter of any one or more of Examples 10-11 may optionally be configured such that the housing includes a base mold having a first portion with a first planar bottom surface and a second portion with a second planar bottom surface. The first and second planar bottom surfaces may form an angle corresponding to a skull contour such that both of the first and second planar bottom surfaces are approximately tangent to a skull surface. The coil frame may be bonded to the first portion of the base mold. The can assembly may be bonded to the second portion of the base mold.

An example (e.g., “Example 13”) of a device may include a housing, at least one lead, stimulator circuitry, a battery, and a battery liner. The housing may include a coil frame and a can assembly. Each of the lead(s) may include a plurality of electrodes. The stimulator circuitry may include a waveform generator configured to generate an electrical waveform and a controller configured to electrically drive the electrical waveform through at least one of the plurality of electrodes. The battery may be configured to provide electrical power to the waveform generator and the controller. The waveform generator, the controller, and the battery may be within the can assembly. The battery liner may be adjacent to at least a portion of a periphery of the battery. The battery liner may include at least one spring configured to press against the periphery of the battery to resist battery movement within the can assembly.

In Example 14, the subject matter of Example 13 may optionally be configured such that the battery liner may be configured to be positioned along at least a width edge of the battery and a length edge of the battery. The spring(s) may include at least one spring is along the width edge of the battery to constrain battery movement in the length direction and at least one spring along the length edge of the battery to constrain the battery movement in the width direction.

In Example 15, the subject matter of any one or more of Examples 13-14 may optionally be configured such that the spring(s) includes an arch spring.

An example (e.g., “Example 16”) of a device may include a housing, at least one lead, stimulator circuitry, a battery and a strain relief. The housing may include a coil frame and a can assembly Each of the lead(s) may include a plurality of electrodes. The stimulator circuitry may include a waveform generator configured to generate an electrical waveform and a controller configured to electrically drive the electrical waveform through at least one of the plurality of electrodes. The battery may be configured to provide electrical power to the waveform generator and the controller. The waveform generator, the controller, and the battery may be within the can assembly. The strain relief may be at a housing-lead interface where the lead(s) enters the housing. The strain relief may include side webs on each side of the lead(s). Each of the side webs may extend from a first position that is near the coil frame and away from the leads to a second position that is near the leads and away from the coil frame. A suture hole may be formed in at least one of the side webs.

In Example 17, the subject matter of Example 16 may optionally be configured to further include a suture hole formed in each of the side webs.

An example (e.g., “Example 18”) of a device may include a housing, at least one lead, stimulator circuitry, a battery, a coil and a flexible tail. The housing may include a base mold having a first portion with a first planar bottom surface, a second portion with a second planar bottom surface, and a third portion with a third planar bottom surface. The first and second planar bottom surfaces may form a first angle corresponding to a skull contour such that both of the first and second planar bottom surfaces are approximately tangent to a skull surface. The second and third bottom surfaces may form a second angle corresponding to skull contour such that the third planar bottom surface is approximately tangent to the skull surface. The coil frame may be bonded to the first portion of the base mold. The can assembly may be bonded to the second portion of the base mold. Each of the lead(s) may include a plurality of electrodes. The stimulator circuitry may include a waveform generator configured to generate an electrical waveform and a controller configured to electrically drive the electrical waveform through at least one of the plurality of electrodes. The battery may be configured to provide electrical power to the waveform generator and the controller. The waveform generator, the controller, and the battery may be within the can assembly. The coil may be electrically connected to the stimulator circuitry. The coil frame may be configured to hold the coil. The flexible tail may be on the third portion of the base mold. The flexible tail and the coil frame may be on opposing sides of the can assembly.

In Example 19, the subject matter of Example 18 may optionally be configured to further include a coating over the housing. Both the coating and the flexible tail may be silicone.

In Example 20, the subject matter of any one or more of Examples 1-19 may optionally be configured such that the lead retention feature includes, for each of the at least one lead, at least one clip configured to receive and grip the proximal lead end to retain the proximal lead end within the housing.

In Example 21, the subject matter of Example 20 may optionally be configured such that the clip(s) for each of the lead(s) includes at least two clips to provide a non-linear lead path for gripping the proximal lead end to retain the proximal lead end within the housing.

In Example 22, the subject matter of Example 21 may optionally be configured such that the clip(s) for each of the lead(s) includes at least three clips, and the non-linear lead path includes a winding path.

In Example 23, the subject matter of any one or more of Examples 1-19 may optionally be configured to further include a coil electrically connected to the stimulator circuitry. The housing may include a coil frame configured to hold the coil, and the coil frame may include the lead retention feature including the at least one clip for each of the at least one lead.

In Example 24, the subject matter of Example 23 may optionally be configured such that the coil frame may include a periphery configured to receive the coil, and a raised central region corresponding to a center of the coil. At least some of the clips may be in the raised central region.

In Example 25, the subject matter of any one or more of Examples 23-24 may optionally be configured such that the clip(s) include raised features separated by a distance corresponding to a diameter of the proximal lead end to receive the proximal lead end between the raised features.

In Example 26, the subject matter of Example 25 may optionally be configured such that the proximal lead end may include an insulator coating that is compressed when the proximal lead end is inserted between the raised features.

In Example 27, the subject matter of any one or more of Examples 1-26 may optionally be configured to further include a strain relief at a housing-lead interface where the at least one lead enters the housing.

In Example 28, the subject matter of Example 27 may optionally be configured such that the strain relief may include side webs on each side of the at least one lead. Each of the side webs may extend from a first position that is near the coil frame and away from the leads to a second position that is near the leads and away from the coil frame.

In Example 29, the subject matter of Example 28 may optionally be configured to further include a suture hole formed in at least one of the side webs.

In Example 30, the subject matter of Example 29 may optionally be configured to further include a suture hole formed in each of the side webs.

In Example 31, the subject matter of any one or more of Examples 1-30 may optionally be configured such that a first portion of a base mold may include at least one post and the coil frame may include at least one aperture corresponding to the at least one post and configured to receive the at least one post when the coil frame is bonded to the second portion of the base mold.

In Example 32, the subject matter of any one or more of Examples 1-31 may optionally be configured to further include silicone over the coil, the coil frame and the can assembly.

An example (e.g., “Example 33”) of a device may include a method for making a medical device, where the method includes forming a base mold with a first planar surface and a second planar surface, bonding a can assembly to the first planar surface, bonding a coil frame to the second planar surface, placing a coil on the coil holder and connecting ends of the coil to stimulator circuitry (the coil holder having a raised center in a center of the coil), routing a proximal lead end for at least one lead through a non-linear path of a lead retention feature, and connecting conductors in the at least one lead to the stimulator circuitry, forming a strain relief boot near an interface of the at least one lead and the coil frame, wherein the strain relief includes at least one suture hole, applying an epoxy over an interface region for the can assembly and the coil assembly, and depositing silicone over the can assembly and the coil frame.

In Example 34, the subject matter of Example 33 may optionally be configured such that the depositing silicone includes forming a flexible tail on an opposing side of the can assembly from the coil frame.

In Example 35, the subject matter of any one or more of Examples 33-34 may optionally be configured such that routing the proximal lead end includes routing the proximal lead in through a winding path in between raised features in a central portion of the coil holder.

In Example 36, the subject matter of any one or more of Examples 33-35 may optionally be configured to further include assembling the can assembly by inserting a battery liner within a feedthrough can. The battery liner may have a shape corresponding to at least a portion of a periphery for a battery. The battery liner may include at least one spring configured to press against the periphery of the battery to resist battery movement within the can assembly. The method may include inserting the battery and a printed circuit board assembly (PCBA) into the feedthrough can. The battery at least partially compresses the at least one spring in the battery liner. The method may further include welding a lid on the can assembly.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example and not limitation, an implantable medical device implanted beneath the skin and over a patient's cranium.

FIG. 2 illustrates, by way of example and not limitation, an external device and headset configured for use to communicate with and/or charge the implantable medical device(s).

FIG. 3 illustrates, by way of example and not limitation, a side view of fully head-located neurostimulation system.

FIG. 4 illustrates, by way of example and not limitation, two implanted devices with leads to cover both sides of the head.

FIG. 5 illustrates, by way of example and not limitation, an implantable medical device with integrated leads.

FIGS. 6A-6B illustrate, by way of example and not limitation, an “epoxy mold view” of a can assembly and a coil frame bonded to a base mold.

FIGS. 7A-7B illustrate, by way of example and not limitation, views of the device after the device is covered with a silicone overmold.

FIG. 8 illustrates, by way of example and not limitation, a partial exploded view of the can assembly connected to the coil frame, and the base mold.

FIG. 9 illustrates, by way of example and not limitation, a closer view of the proximal lead ends held in place with the lead retention clips in the coil frame.

FIG. 10 illustrates, by way of example and not limitation, a closer view of electrical connections to feedthroughs.

FIG. 11 illustrates, by way of example and not limitation, an exploded view of the can assembly.

FIGS. 12A-12D illustrate, by way of example and not limitation, views of some components of the can assembly.

FIG. 13 illustrates, by way of example and not limitation, an exploded view of the lid and feedthrough can with electronics and battery contained within the can.

FIG. 14 illustrates, by way of example and not limitation, a view of the lid welded to the feedthrough can.

FIG. 15 illustrates, by way of example and not limitation, feedthrough cutouts in the feedthrough can.

FIG. 16 illustrates, by way of example and not limitation, the electrical connection of the lead wires to the lead feedthrough terminals.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

The implantable device may be a subcutaneous neurostimulator. The neurostimulator may include an implantable pulse generator and two leads integrated into the assembly of the implanted pulse generator (IPG). One lead may be longer than the other for choice in location placement for best fit and function. There are multiple electrode segments on each lead, which enables multiple pulse programming options and reprogramming options if there were to be lead pullback. The integration of the leads avoids the added size and complexity of a lead to IPG connection interface. The lead length is intended to reach the targeted nerves without having excessive lead length that could interfere with patient comfort. Multiple electrodes on each lead allows for multiple pulse delivery combinations for optimized pulse delivery to targeted area and adjustment to delivery if there were to be a shift in electrode location. The implantable device may include one or more of the following features that aid in the durability of the assembly and aid its implantation.

For example, the device may be able to accommodate rechargeable batteries of varying sizes due to large manufacturing tolerances. The device may include springs (e.g., arch-type) integrated into a frame (e.g., polymer frame). The polymer frame may support the printed circuit board and isolate the battery. There are arch type springs that hold the rechargeable battery. These springs are integrated into the internal frame made from a polymer. The batteries generally have a rectilinear foot print, providing the battery with a length and width. At least one spring is on a width edge (e.g., horizontal X direction) of the battery to constrain the battery in the length direction (e.g., vertical (Y) direction), and at least one spring is on a length edge (e.g., vertical (Y) direction) of the battery to constrain the battery in the width direction (e.g., horizontal X direction). These springs constrain the battery in both in the vertical (y) horizontal (x) direction, accommodating different sizes of battery due to manufacturing tolerances.

The device may have a strain relief for the leads at the IPG body interface for bend durability of the leads that are integrated with the IPG. The strain relief may be molded into the IPG body at the lead to IPG interface. This strain relief protects the lead from kinking, or otherwise excessively bending, at a vulnerable location to avoid flex fatigue and lead damage that may occur from flex fatigue.

The device may include suture holes near the lead strain relief that can be used with suture to constrain the IPG in the intended location. The suture holes may be molded or otherwise incorporated into the silicone over-molding of the IPG. The suture holes may be located near the lead strain relief. This location can be used to stabilize the IPG in the pocket and anchor the base of the lead at the same time if desired.

The device may have a lead retention feature built into the IPG body to lock and retain the leads in a serpentine manner, which locks and restrains the proximal ends of the integrated leads in the IPG. The device may have a hermetically-welded IPG assembly (e.g., “can”) to house electronics, and a coil frame to hold the communication and charging coil. The coil may be a D-shaped coil. The coils frame may include the lead retention features. During assembly, the proximal ends of the integrated leads are attached to the feedthrough pins of the hermetic enclosure. The lead body is clipped into features of the coil frame to hold the proximal end of the lead body securely. These clip features are in a staggered location that force the lead body to have a serpentine configuration. This serpentine configuration creates a strong friction hold of the lead when pulled in an axial direction away from the IPG. This serpentine lead locking feature creates a very robust grip on the leads keeping them attached to the IPG. Without this feature, the leads could be detached much more easily during implant or at end-of-life extraction.

The device has an angled shape. The coil frame may be mechanically locked in place with respect to the hermetically-welded IPG assembly. This frame may be mechanically locked in place at the desired angle to make a conforming IPG shape. The mechanical lock may be created by molding an epoxy interface between the hermetically-welded IPG assembly (e.g., a titanium can) and the coil frame. Features in the hermetically-welded IPG assembly and the coil frame may be filled with the epoxy, which creates the interlock when the epoxy cures. The angle provides a comfortable fit to the skull. The coil frame holds the communication and charging coil as well as the proximal end of the leads. Mechanically and rigidly attaching the coil frame to the IPG can protects the delicate cables and wires that are attached to the feedthrough pins from flex fatigue and tension.

The device may include a short flexible “tail” molded into the IPG body, which may rest on the nuchal ridge after implant and may give a softer transition to the more rigid components of the IPG. Each of the short flexible tail, the hermetically-welded IPG assembly and the coil frame may have planar bottom surface, designed to provide a desired angle between the short flexible tail and the hermetically-welded IPG assembly, and provide a desired angle between the hermetically-welded IPG assembly and the coil frame to generally follow the rounded shape of the skull. The top surfaces of the short flexible tail, the hermetically-welded IPG assembly and the coil frame generally follow the bottom surfaces to avoid the subcutaneously implanted IPG from significantly raising or “tenting” the skin.

By way of example and not limitation, a medical device system may include implantable medical device(s) and an external device configured for use to communicate with and/or charge the implantable medical device(s). More particularly, the system may include a fully head-located neurostimulator(s) designed for the treatment of chronic head pain. The system may be configured to provide neurostimulation therapy for chronic head pain, including chronic head pain caused by migraine and other headaches, as well as chronic head pain due other etiologies. For example, the system may be used to treat chronic head and/or face pain of multiple etiologies, including migraine headaches; and other primary headaches, including cluster headaches, hemicrania continua headaches, tension type headaches, chronic daily headaches, transformed migraine headaches; further including secondary headaches, such as cervicogenic headaches and other secondary musculoskeletal headaches; including neuropathic head and/or face pain, nociceptive head and/or face pain, and/or sympathetic related head and/or face pain; including greater occipital neuralgia, as well as the other various occipital neuralgias, supraorbital neuralgia, auriculotemporal neuralgia, infraorbital neuralgia, and other trigeminal neuralgias, and other head and face neuralgias.

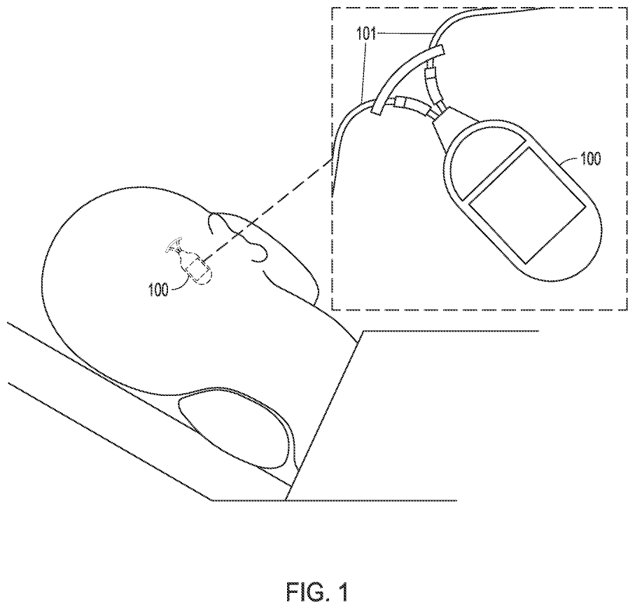

FIG. 1 illustrates, by way of example and not limitation, an implantable medical device 100 implanted beneath the skin and over a patient's cranium. The device may be referred to as a fully-head-located implantable device. The device 100 is illustrated as being implanted behind and above the ear. The implantable medical device 100 may include one or more leads 101 that may be subcutaneously tunneled to a desired neural target. The lead(s) may be integral to the device 100, as they have proximal lead ends that extend into housing of the device where electrical connections are made between stimulator circuitry and electrodes on the lead. When the medical device has integral leads, the leads are not removably connected to the device. The number of electrodes and spacing may be such as to provide therapeutic stimulation over any one or any combination of the supraorbital, parietal, and occipital region substantially simultaneously. The implantable medical device 100 may be configured to independently control each electrode to determine whether the electrode will be inactive or configured as a cathode or an anode. One or more electrodes on the lead(s) may be configured to function as an anode, and one or more electrodes on the lead may be configured to function as a cathode. For example, bipolar neuromodulation may be delivered using one or more anodes and one or more cathodes on the lead(s). A clinician may program the electrode configurations to provide a neuromodulation field that captures a desired neural target for the therapy.

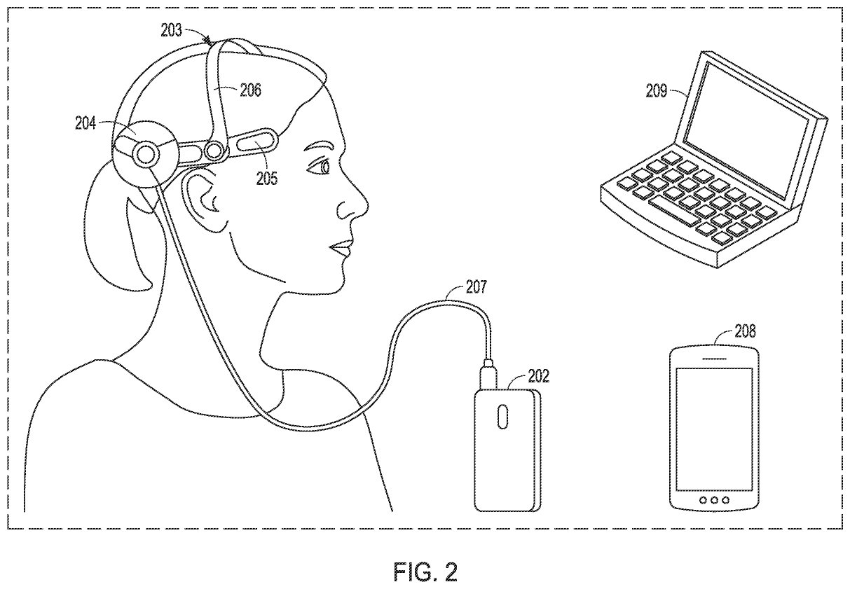

FIG. 2 illustrates, by way of example and not limitation, an external device 202 and headset 203 configured for use to communicate with and/or charge the implantable medical device(s). The headset 203 may include an external coil 204 used to provide the communication/charging functions, and the headset 203 may be configured to position the external coil over an implantable medical device. For example, the headset 203 may include an adjustable frame 205 on each side of the head that can rotate about a point on a main headset frame 206, and may be configured to provide additional degrees of motion (e.g., sliding or pivoting motion) with respect to the main headset frame 206. These adjustable frames may be used to position the external coils 204 over the implantable medical devices when the main headset frame 206 is worn. The external device 202 may be electrically connected to the external coil 204 via a cable 207. In some embodiments, the external device 202 may be wirelessly connected to the headset 203. The headset may be configured to wirelessly receive power from the external device and to transfer power from the external coil to the implanted device(s). The external device may communicate, using wireless or wired communication technology, with other external devices such as a phone 208 or programmer 209.

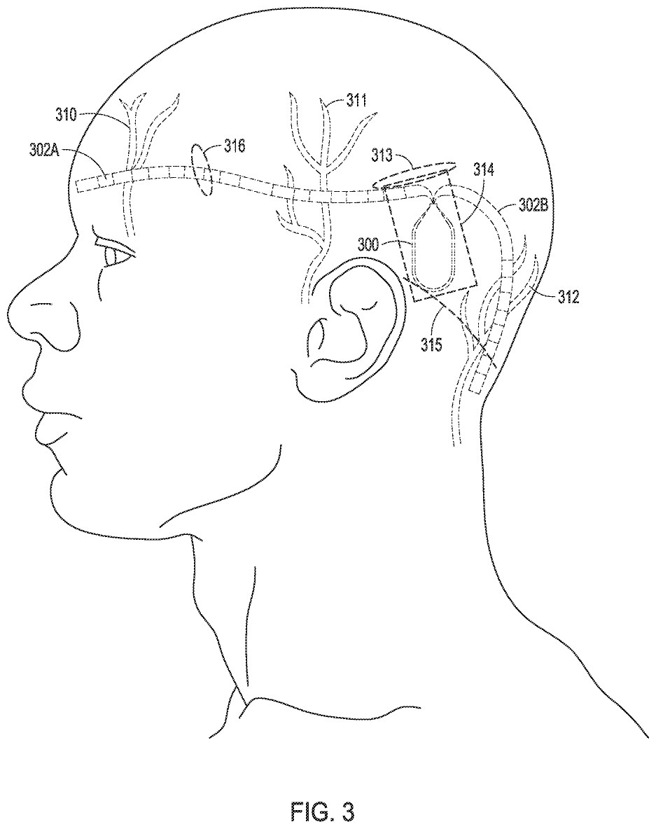

FIG. 3 illustrates, by way of example and not limitation, a side view of fully head-located neurostimulation system. Also illustrated are the supraorbital nerve 310, the auriculotemporal nerve 311 and the occipital nerve 312. The illustrated system includes a medical device (e.g., pulse generator) 300 and both a first lead 302A and a second lead 302B attached to the medical device 300. The medical device 300 and leads 302A and 302B are configured for use in stimulating the supraorbital nerve 310 toward the front of the head and the occipital nerve 312 toward the back of the head.

The patient may have had a period of trial neurostimulation, which is standard in traditional neurostimulator evaluations but is optional here. The actual permanent implant may occur in a standard operating suite with appropriate sterile precautions. By way of example and not limitation, the patient may be prepped and draped. The patient may be administered prophylactic antibiotics, local anesthetic, and sedation. The patient may be placed in a supine position with a head of the bed elevated to approximately thirty degrees. The patient's head may be turned to better access the intended implant location. While the implantable medical device may be positioned subcutaneously anywhere, it may be positioned above and behind the ear in this illustrated embodiment. Thus, a first incision 313 of sufficient length (approximately 4-6 cm) is made to a depth sufficient to reach the subcutaneous layer. A pocket 314 to accept the medical device 300 is fashioned by standard dissection techniques. The pocket 314 may be directed below the incision. The pocket 314 may be angled depending on the desired orientation of the medical device. For example, the pocket 314 may be angled posteriorly, as illustrated. The pocket 314 may be 10-20% larger than the medical device 300 to allow for a comfortable fit and no undue tension on the overlying skin and/or incision. The first incision 313 may be made and the pocket 314 formed so that the implantable medical device abuts against the nuchal ridge 315 when fully inserted into the pocket 314. The first incision 313 should not interfere with the implanted medical device 300. The present subject matter may use template(s) to help make the incision in a desired location.

A second incision 316 may be made to the subcutaneous layer at a point above and anterior to the pinna of the ear in the temple region to assist with subcutaneously routing the first lead 302A. The first lead 302A may be passed from the medical device 300 in the pocket 314 to the second incision 316, and then passed from the second incision 316 to its final subcutaneous position over supraorbital nerves 310. The second lead 302B may be passed from the medical device 300 in the pocket 314 back toward the occipital nerve 312. The medical device 300 may be inserted into the pocket 314 either before or after the leads 302A and/or 302B are tunneled to their final subcutaneous position to deliver therapy.

Tubular introducer(s) with a plastic-peel away shell may be used to assist with lead placement. However, other techniques may be used to subcutaneously tunnel the leads to their final placement to deliver the neurostimulation therapy. Following the entire placement of the complete system, including the medical device and both leads and suturing, the medical device may be powered-up and its circuits checked. Upon recovery from anesthesia the system may be turned on for the patient with a portable programmer and the multiple parameters for the system may be programmed to provide a desired therapy for the patient.

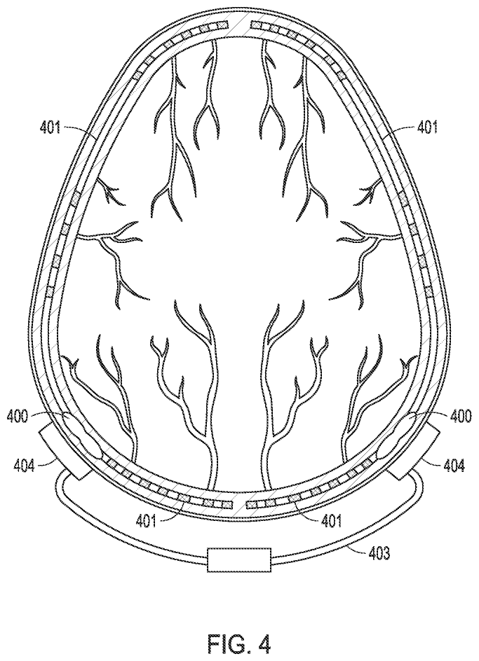

FIG. 4 illustrates, by way of example and not limitation, two implanted devices 400 with leads 401 to cover both sides of the head with one of the devices on the left side of the head and the other on the right side of the head, and a charging/communication headset 403 disposed about the cranium. The headset 403 may include right and left coupling coil enclosures that each contain a coil for coupling to the respective coils in the implants. The coil enclosures may interface with a main charger/processor body which may contain processor circuitry and batteries for both charging the internal battery in the implantable medical devices 400 and also communicating with the implanted devices. Thus, in operation, when a patient desires to charge their implanted devices 400, all that is necessary for some embodiments is to place the headset about the cranium with the coils 404 in close proximity to the respective implanted devices 400. In some embodiments, such placement may automatically initiate charging; whereas in other embodiments, the user may initiate charging using an external device. When the headset 403 is worn by a patient, the headset coils (transmit coils) 404 are placed in proximity to the corresponding receive coil in each respective body-implanted implantable medical device 400. The headset 403 may include telemetry circuitry, a controller, a battery, and a Bluetooth wireless interface. The headset 403 may communicate with a personal device such as a smartphone or tablet (e.g., via the Bluetooth interface), for monitoring and/or programming operation of the two implantable medical devices.

The implantable medical device 400 may include a rechargeable battery, an antenna (e.g., coil), and at least one Application Specific Integrated Circuit (ASIC), along with the necessary internal wire connections amongst these related components, as well as to the incoming lead internal wires. These individual components may be encased in a can made of a medical grade metal. The battery may be connected to the ASIC(s) via a connection that is flexible. The overall enclosure for the battery, antenna and ASIC(s) may have a very low flat profile with two lobes, one lobe for housing the ASIC(s) and one lobe for housing the battery. The antenna may be housed in either of the lobes or in both lobes. The use of the two lobes and the flexible connection between the ASIC(s) and the battery allows the implanted device to conform to the shape of the human cranium when subcutaneously implanted.

The ASIC(s) and lead may be configured to independently drive each of the electrodes using a neuromodulation signal in accordance with a predetermined program. The programmed stimulation may be defined using parameters such as one or more pulse amplitudes, one or more pulse widths and one or more pulse frequencies. Other parameters may be used for other defined waveforms, which may but does not necessarily use rectilinear pulse shapes. Once the program is loaded and initiated, a state machine may execute the particular programs to provide the necessary therapeutic stimulation. The ASIC(s) may have memory and be configured for communication and for charge control when charging a battery Each electrode may be individually turned off (inactive), or individually activated and designated as an anode or a cathode.

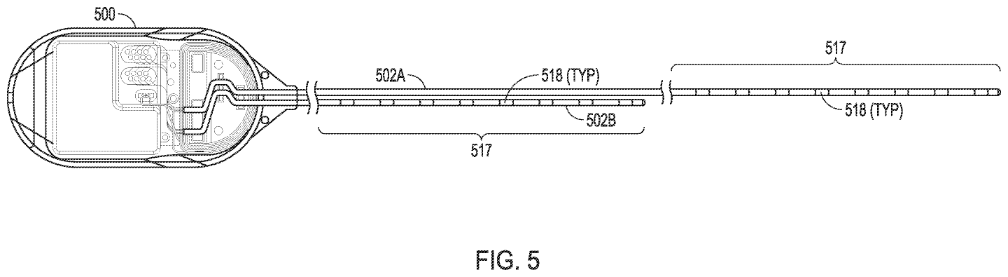

FIG. 5 illustrates, by way of example and not limitation, an implantable medical device 500 with integrated leads 502A and 502B. The illustrated integrated leads include a longer lead 502A configured for use to deliver neuromodulation to the supraorbital nerves and a shorter lead 502B configured for use to deliver neuromodulation to the occipital nerves (see, by way of example, FIG. 3). The length of each lead is determined to reach the neural target without excess lead. Each may include an electrode array 517 with a plurality of electrodes 518. In the illustrated example, the number of electrodes in each array is eight electrodes. However, the arrays may include more or less electrodes. The electrodes 518 may be independently controlled to allow the therapy to be programmed to use the appropriate electrodes to capture the targeted nerves. That is, the electrode arrays are long enough so that, after the implantation procedure, at least some of the electrodes are operably near the targeted nerves to deliver therapeutic neuromodulation energy. The programming of the therapy can determine which electrodes are active for delivering the energy and which electrodes are inactive. Additionally, the programming can determine the polarity of the active electrodes. For example, the therapy may be programmed to use one or more anodes and one or more cathodes selected from the electrodes 518 in the electrode array 517.

The lead(s) 502A and 502B are integral to the device 500, as they have proximal lead ends that extend into housing of the device where electrical connections are made between stimulator circuitry and the lead conductors. The leads are not removably connected to the device. Rather, conductors in the lead are welded or otherwise connected to feedthrough conductors within a hermetically-sealed housing to provide a permanent electrical connection for the operably life of the device. In contrast, implantable devices that use removable leads may have connector blocks or other electrical contact in which the leads may be removably inserted to make the electrical connection to the device.

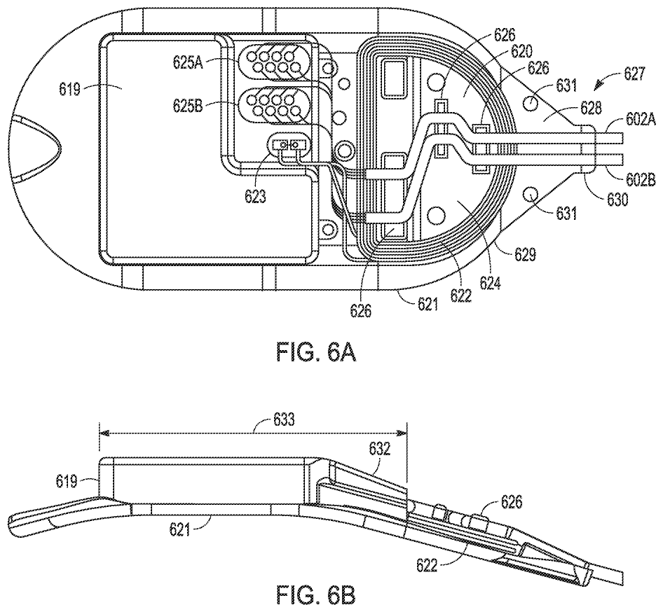

FIGS. 6A-6B illustrate, by way of example and not limitation, an “epoxy mold view” of a can assembly 619 and a coil frame 620 bonded to a base mold 621. A coil (e.g., D-shaped coil) 622 is held on the coil frame 620. The flat edge of the D-shaped coil 622 may be adjacent to the can assembly 619, which enables the device to have a more compact design. Ends of the coil 622 are terminated at the coil feedthrough terminals 623, which provides an electrical connection to the stimulator circuitry within the can assembly. The leads have electrical conductors that extend from the electrodes and are terminated at the lead feedthrough terminals 625A, 625B, which provides an electrical connection to the stimulator circuitry within the can assembly. The integrated leads 602A, 602B have proximal lead ends 624 that extend into the device housing, and are retained by a lead retention feature on the coil frame 620. The lead retention feature may hold each lead within the device in a non-linear path such as a winding path, which holds the integrated leads and restrains the leads from being pulled out of the device during normal use. For example, the illustrated lead retention feature is built into the body of the device to lock and retain the leads in a serpentine manner, which locks and restrains the proximal ends of the integrated leads within the device housing. The illustrated lead retention feature includes clips in which the proximal lead ends may be inserted. For example, each proximal lead end may include three clips. The coil frame 620 may include a periphery configured to receive the coil 622, and a raised central region corresponding to a center of the coil where the clips may be provided to provide a non-linear lead path for gripping the proximal lead end. Each clip may include raised features, raised above a floor of the coil frame, separated by a distance corresponding to a diameter of the proximal lead end to receive the proximal lead end between the raised features. The proximal lead end may include an insulator coating, which may be compressed between the raised features without breaking the coating when the proximal lead end is inserted.

The device may have a strain relief 627 for the leads at a housing-lead interface for bend durability of the integrated leads. The strain relief may be molded into the device housing. This strain relief protects the lead from kinking, or otherwise excessively bending, at a vulnerable location to avoid flex fatigue and lead damage that may occur from flex fatigue. The strain relief may include side webs 628 on each side of the leads. Each of the side webs 628 may extend from a first position 629 that is near the coil frame and away from the leads to a second position 630 that is near the leads and away from the coil frame. At least one suture hole 631 may be formed in at least one of the side webs. In the illustrated embodiment, each web 628 incudes suture hole 631. The suture holes 631 near the lead strain relief 627 may be used with suture to constrain the device in the intended location. This location can be used to stabilize the device in the pocket and anchor the base of the lead at the same time if desired. The suture holes 631 may be molded or otherwise incorporated into the silicone over-molding of the device.

FIG. 6B illustrates an epoxy 632 over an interface between the can assembly 619 and the coil frame which is holding the coil 622. The coil frame near the interface and the can assembly near the interface may have features for enhancing an ability of epoxy to fix the coil frame to the can assembly. The epoxy may be provided over the interface and hardens to fix the coil frame to the can apertures in each of the coil frame and the can assembly. The deposited epoxy may at least partially fill the apertures, which enhances the ability of the epoxy to fix the coil frame to the can assembly when the epoxy hardens. Other features may be used such as posts, grooves, ridges, and the like. The distance 633 from one edge of the can assembly 619 to an edge of the epoxy may be about 1.3 inches. The epoxy may completely cover the lead conductors and coil wires.

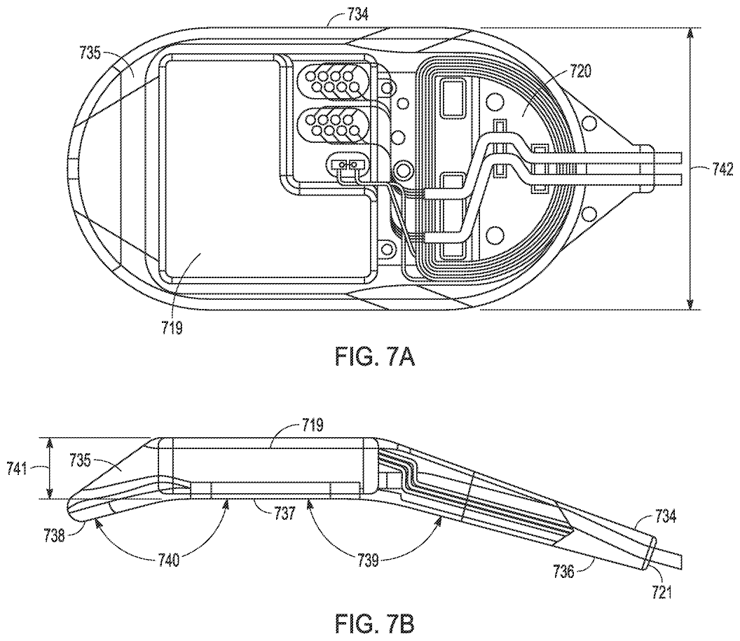

FIGS. 7A-7B illustrate, by way of example and not limitation, views of the device after the device is covered with a silicone overmold. The silicone 734 may completely cover the entire device, including the epoxy (see FIG. 6B, 632). The silicone 734 may form a flexible tail 735. The flexible tail 735 and the coil frame 720 are on opposing sides of the can assembly 719. The base mold 721 may have a first portion 736 with a first planar bottom surface, a second portion 737 with a second planar bottom surface, and a third portion 738 with a third planar bottom surface. The bottom surfaces for the first and second portions 736 and 737 form a first angle 739 and the bottom surfaces for the second and third portions 737 and 738 form a second angle 740. The angles and bottom surfaces generally correspond to a skull contour such that the planar bottom surfaces are approximately tangent to a skull surface. Each of the first and second angles 739 and 740 may be within a range between 160-170 degrees. The coil frame for holding a coil may be bonded on a first portion 736 of the base mold. The can assembly 719 for housing the stimulator circuitry may be bonded on the second portion 737 of the base mold. The silicone for the flexible tail 735 may be formed and bonded on a third portion 738 of the base mold 721. The thickness 741 of the device may be between 0.2 and 0.3 inches (e.g., approximately 0.26 inches), and the width 742 of the device may be within a range between 1 inch and 1.5 inches (e.g., approximately 1.22 inches).

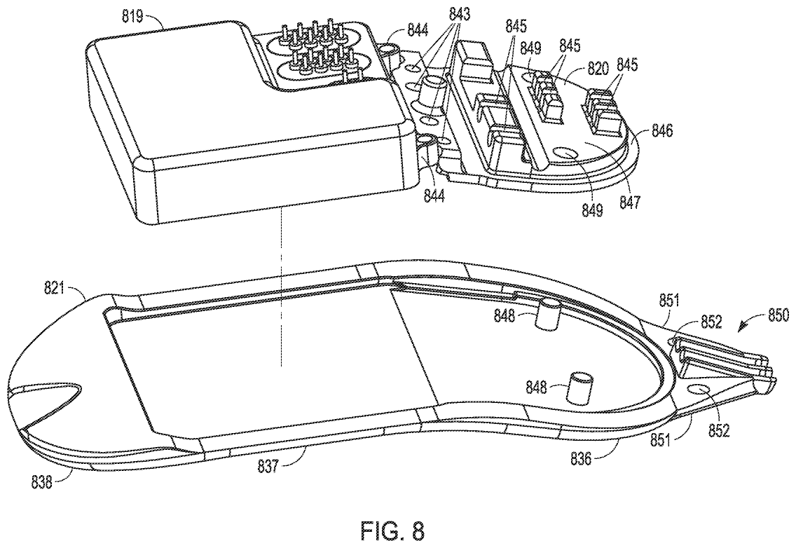

FIG. 8 illustrates, by way of example and not limitation, a partial exploded view of the can assembly 819 connected to the coil frame 820, and the base mold 821. The housing of the device includes the can assembly 819, the coil frame 820 and the base mold 821, along with the epoxy overmold. The can assembly 819 may house stimulator circuitry and a battery configured to provide electrical power to the stimulator circuitry. The stimulator circuitry may include a waveform generator configured to generate an electrical waveform and a controller configured to electrically drive the electrical waveform through at least one of the plurality of electrodes. The coil frame 820 is configured to hold a coil, which will be electrically connected to the electrical circuitry within the can assembly 819. As illustrated in FIG. 6B, an epoxy is deposited over an interface between the coil frame and the can assembly to fix the coil frame to the can assembly. Each of the coil frame 820 near the interface and the can assembly 819 near the interface may have features for enhancing an ability of epoxy to fix the coil frame 820 to the can assembly 819 when hardened. For example, the features for enhancing the ability of the epoxy to fix the coil frame to the can assembly may include coil frame apertures 843 and the can assembly apertures 844. The epoxy may flow into and at least partially fill these apertures and then harden to fixate the coil frame to the can assembly. Other features, such as grooves, ridges and posts may be used to enhance the fixing capability of the epoxy.

The device housing may include a lead retention feature configured to retain the proximal lead end of each lead. For example, the coil frame 820 may be configured with the lead retention feature. The lead retention feature may include at least one clip 845 for each lead. The clip 845 may be configured to receive and grip the proximal lead end. Thus, the clip 845 firmly holds the integral lead from being pulled out of the housing during normal use. The lead retention feature may include, for each lead, at least two clips 845 to provide a non-linear lead path for gripping the proximal lead end. The non-linear lead path further increases the retention of lead, as any force that pulls out on the lead will have lateral force components against the clips. It is difficult to pull leads, with insulator coatings, through the clips at the angles corresponding to the winding lead path. At least three clips 845 may be used for each lead to provide a winding lead path. The coil frame 820 may include a periphery 846 configured to receive the coil, and a raised central region 847 corresponding to a center of the coil such that the coil wraps around the raised central region 847. At least part of the lead retention feature (e.g., at last some of the clips) 845 may be in in the raised central region 847 to provide a non-linear lead path for gripping the proximal lead end.

The first portion 836 of the base mold 821 may include at least one post 848 and the coil frame includes at least one aperture 849 configured to receive the at least one post 848 when the coil frame is bonded to the first portion 836 of the base mold 821. The base mold 821 may include a strain relief portion 850 that corresponds to a lead strain relief (e.g., see FIG. 6A, 627). The strain relief portion 850 of the base mold may include side webs 851, and a suture hole 852 in each of the side webs 851.

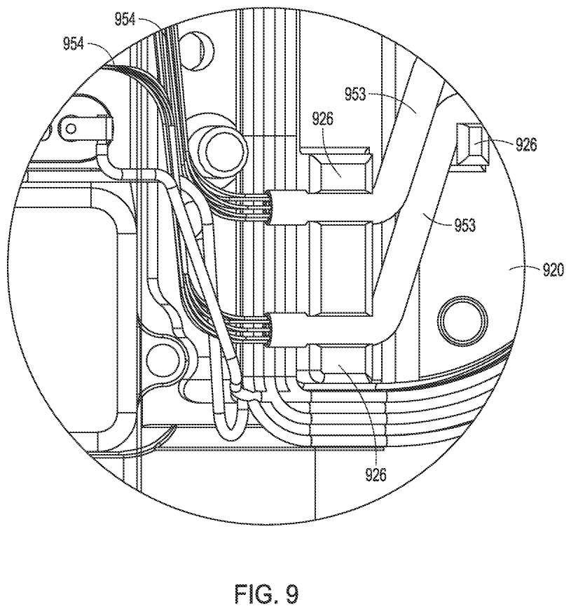

FIG. 9 illustrates, by way of example and not limitation, a closer view of the proximal lead ends 953 held in place with the lead retention clips 926 in the coil frame 920. Each lead includes a plurality of conductors 954 used to provide independent connections to the electrodes in the lead. Each clip 926 may include raised features separated by a distance corresponding to a diameter of the proximal lead end 953 to receive the proximal lead end between the raised features. The lead, including the proximal lead end, may include an insulator coating. The coating is flexible but tough such that it is resistant to tearing. The insulator may be compressed when the proximal lead end is inserted between the raised features of the clip 926, and may partially expand between the clips to resist the proximal lead end from being moved between the raised features of the clips.

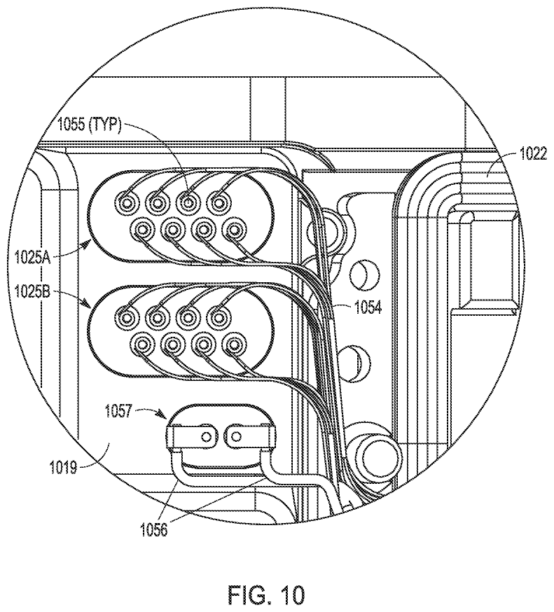

FIG. 10 illustrates, by way of example and not limitation, a closer view of electrical connections to feedthroughs. The electrical conductors 1054 that extend to and are terminated at the lead feedthrough terminals 1025A, 1025B, which provides an electrical connection to the stimulator circuitry within the can assembly 1019. The figure illustrates eight wires, corresponding to the eight electrodes in each lead, being terminated in eight corresponding feedthrough pins 1055. The coil 1022 has coil ends 1056 that may be terminated at compression feedthrough terminals 1057. The stimulator circuitry within the can assembly 1019 is operably connected to the coil 1022 and to each electrode in each lead using these feedthrough terminals 1025A, 1025B, 1057.

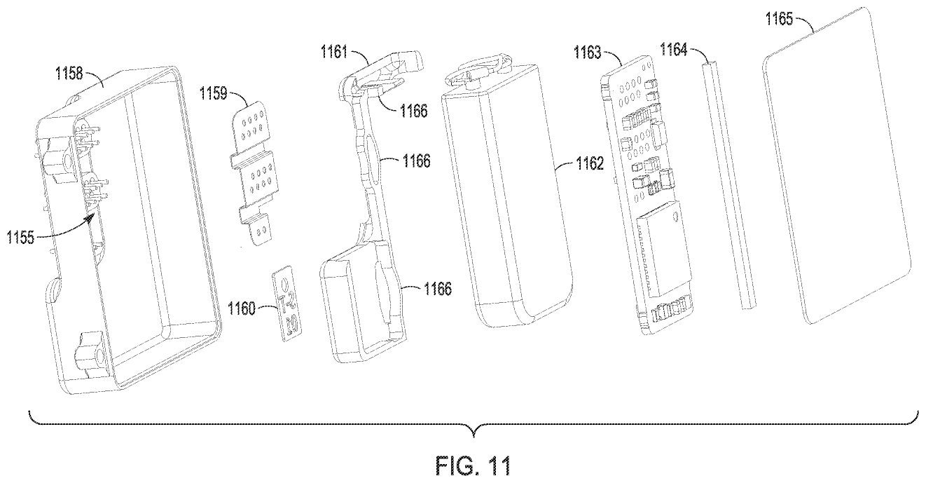

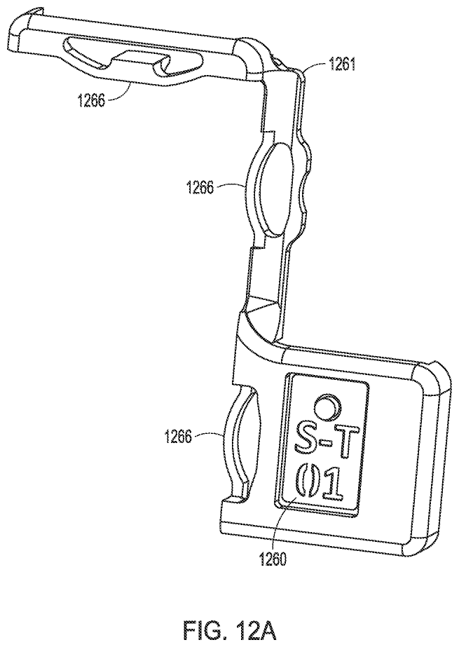

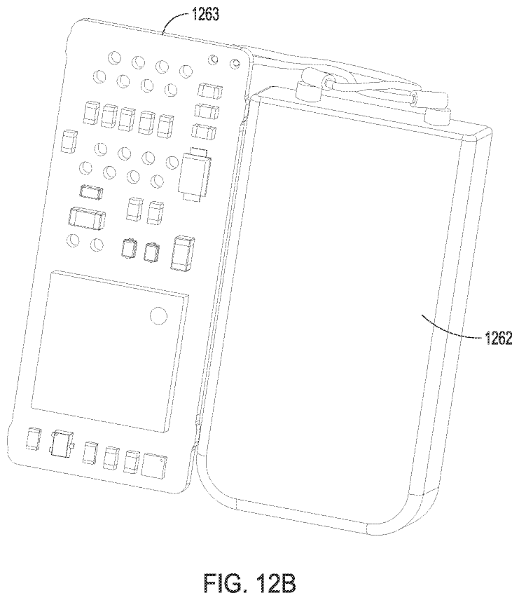

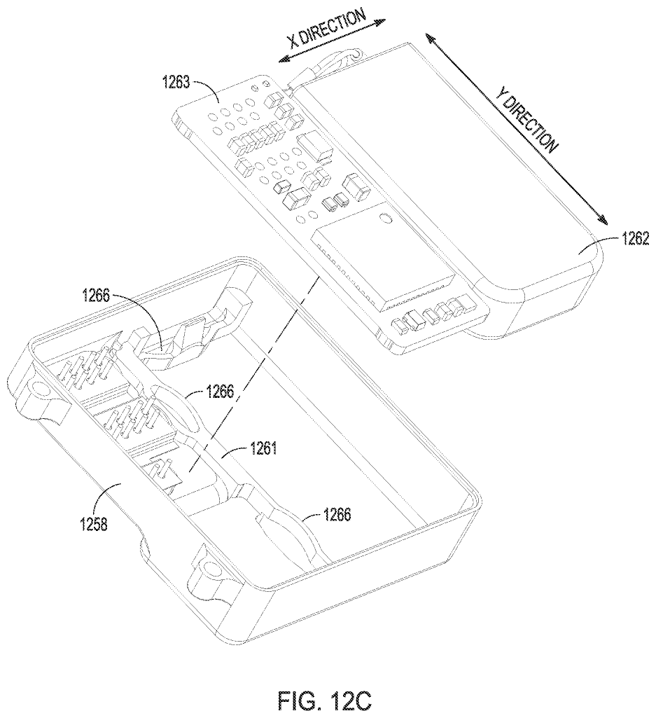

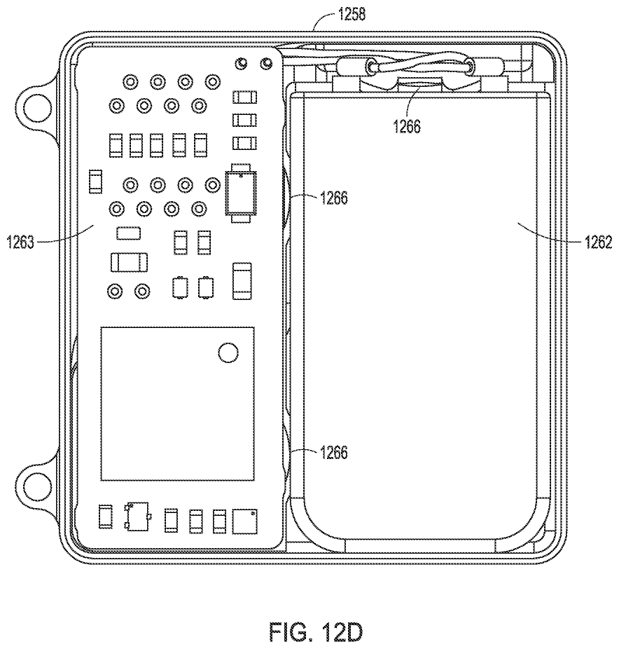

FIG. 11 illustrates, by way of example and not limitation, an exploded view of the can assembly. The illustrated assembly includes a feedthrough can 1158, a feedthrough insulator 1159, an X-Ray identifier 1160, a battery liner 1161, a battery 1162, a printed circuit board assembly (PCBA) 1163, a desiccant 1164, and a can lid 1165. The feedthrough can 1158 may include feedthrough pins 1155 that extend through corresponding holes in the feedthrough insulator 1159. The X-Ray identifier 1160 includes identifying information for the device that can be easily viewed by an x-ray. For example, a model number may be created using material that is opaque to x-rays such that the model number is easily discernible in an x-ray image. The liner 1161 is sized to frame the battery 1162. The liner or frame may support the printed circuit board and isolate the battery. The liner includes spring 1166, such as arch springs, that press against a periphery of the battery 1162 to limit motion in both length and width directions for the battery. For example, the device may be able to accommodate rechargeable batteries of varying sizes due to large manufacturing tolerances. The battery 1162 may generally have a rectilinear foot print, providing the battery with a length and width. At least one spring 1166 may be on a width edge (e.g., horizontal X direction) of the battery to constrain the battery in the length direction (e.g., vertical (Y) direction), and at least one spring may be on a length edge (e.g., vertical (Y) direction) of the battery to constrain the battery in the width direction (e.g., horizontal X direction). These springs constrain batteries of different sizes both in the vertical (y) horizontal (x) direction. X and Y directions for the battery are illustrated in FIG. 12C.

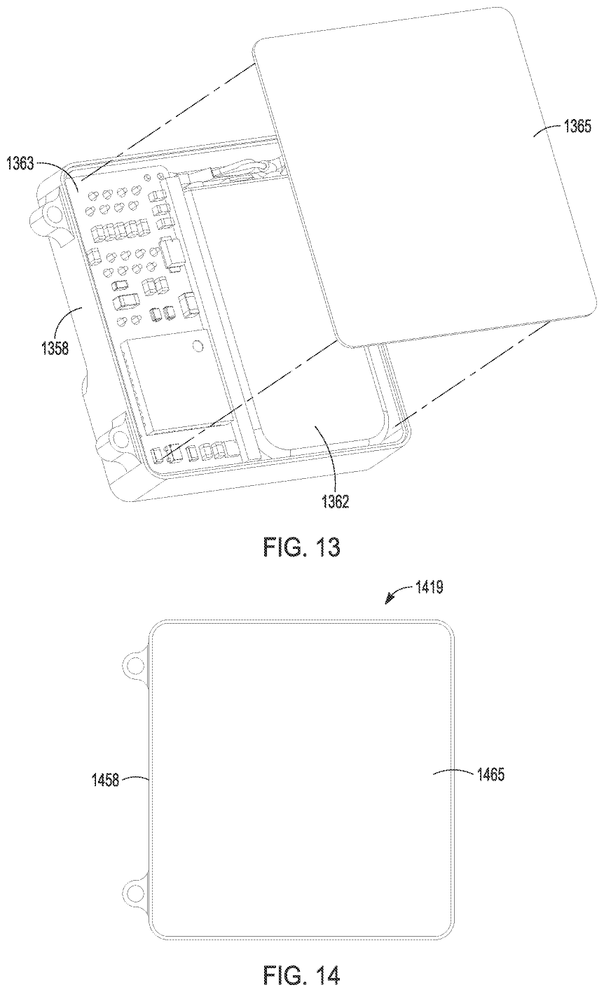

FIGS. 12A-12D illustrate, by way of example and not limitation, views of some components of the can assembly. FIG. 12A illustrates the liner with the X-Ray identifier. The liner shows the arch spring designed to press against the battery and accommodate variations in battery sizes that occur because of large manufacturing tolerances for the batteries. FIG. 12B illustrates the PCBA 1263 connected to the battery 1262. FIG. 12C illustrates the liner 1261 in the feedthrough can 1258, and the PCBA and battery being placed into the feedthrough can. FIG. 12D illustrate the PCBA and battery in the feedthrough can, with the springs 1266 of the liner pressing against the periphery/sides of the battery 1262. FIG. 13 illustrates, by way of example and not limitation, an exploded view of the lid 1365 and feedthrough can 1358 with electronics and battery contained within the can, and FIG. 14 illustrates, by way of example and not limitation, a view of the lid 1465 welded to the feedthrough can 1458. The assembled can assembly 1419 may be mounted on the base mold (see FIG. 8).

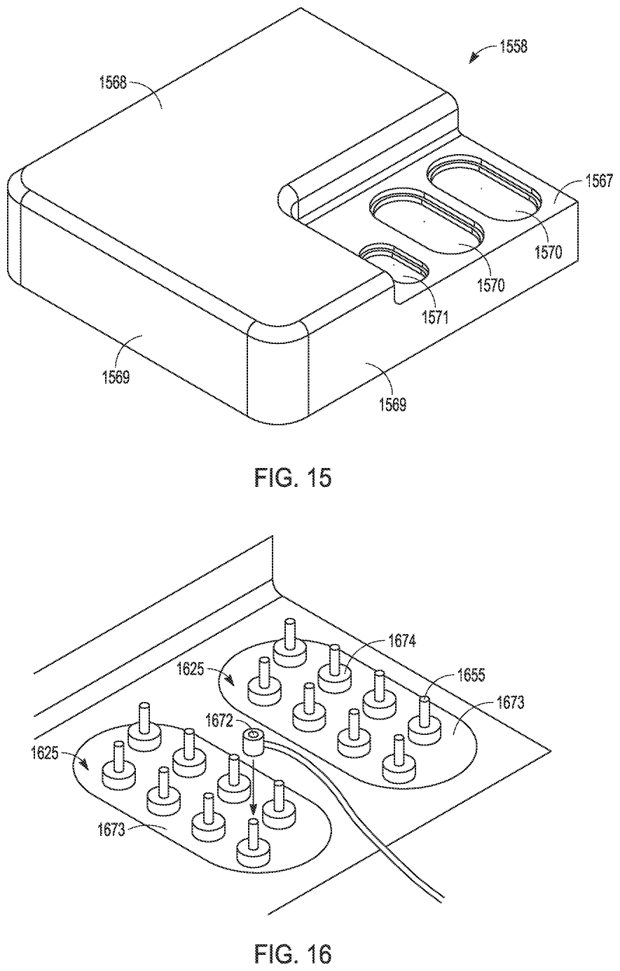

FIG. 15 illustrates, by way of example and not limitation, feedthrough cutouts in the feedthrough can 1558. A recessed portion 1567 is positioned at a corner of the top side 1568. A side wall 1569 extends downward from the edges of the top side 1568. The recessed portion 1567 of the top side 1568 includes larger cut-outs 1570 and a smaller cut-out 1571. In one embodiment, cut-outs 1570 and 1571 are generally oval with parallel long axes. Larger cut-outs 1570 have a length of approximately 0.6 inches and a width of approximately 0.3 inches with smaller cut-out 1571 having a length of approximately 0.2 inches and a width of approximately 0.1 inches. Cut-outs 1570 and 1571 are spaced approximately 0.04 inches apart with the long axes of the cut-outs extending parallel to each other. The cut-outs facilitate feedthroughs which connect the components housed within the can assembly to the leads and the coil, which are external to the can. Any type of feedthrough that provides a hermetic seal for a feedthrough can be facilitated.

FIG. 16 illustrates, by way of example and not limitation, the electrical connection of the lead wires to the lead feedthrough terminals 1625. The coil and the leads are connected to the internal components within the can assembly via the feedthrough pin 1655. A metal sleeve 1672 may be welded to the end of each of the wires coming from the external leads. The metal sleeves are made of any metal appropriate for making good electrically conductive connections (e.g., a platinum-iridium alloy) The metal sleeves 1672 are sized such that they will fit onto the conductive metal pin 1655. The sleeve 1672, with the wire attached, may be lowered onto the appropriate pin 1655 so that the pin is inserted into the center of the sleeve. The sleeve 1672 stays separated from the metal flat portion 1673 of the feedthrough by the insulating glass or ceramic ring 1674. The sleeve 1672 may be spot welded or laser welded to the pin to secure the sleeve on the pin and help create a reliable electrical connection between the sleeve 1672 and the pin 1655. It can be seen from the above that the connections of the lead wires to the feedthrough pins are permanent connections for the operating life of the medical device. The proximal end of the leads is covered by an epoxy and a silicone coating.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using combinations or permutations of those elements shown or described.

The above description is intended to be illustrative, and not restrictive. For example, the above-described examples (or one or more aspects thereof) may be used in combination with each other. Other embodiments may be used, such as by one of ordinary skill in the art upon reviewing the above description. The scope of the invention should be determined with reference to the appended claims, along with the full scope of equivalents to which such claims are entitled.

What is claimed is:

1. A device, comprising:
a housing including a lead retention feature;
at least one lead, each of the at least one lead including a plurality of electrodes;
stimulator circuitry, including a waveform generator configured to generate an electrical waveform and a controller configured to electrically drive the electrical waveform through at least one of the plurality of electrodes;
a battery configured to provide electrical power to the stimulator circuitry;
wherein the stimulator circuitry and the battery are within the housing, each of the at least one lead including a plurality of electrical conductors extending from a proximal lead end to provide electrical connections to the plurality of electrodes, the plurality of electrical conductors being electrically connected to the stimulator circuitry, the proximal lead end being retained within the housing by the lead retention feature.

2. The device of claim 1, wherein the lead retention feature includes, for each of the at least one lead, at least one clip configured to receive and grip the proximal lead end to retain the proximal lead end within the housing.

3. The device of claim 2, wherein the at least one clip for each of the at least one lead includes at least two clips to provide a non-linear lead path for gripping the proximal lead end to retain the proximal lead end within the housing.

4. The device of claim 3, wherein the at least one clip for each of the at least one lead includes at least three clips, and the non-linear lead path includes a winding path.

5. The device of claim 2, further comprising a coil electrically connected to the stimulator circuitry, the housing including a coil frame configured to hold the coil, wherein the coil frame includes the lead retention feature including the at least one clip for each of the at least one lead.

6. The device of claim 5, wherein the coil frame includes a periphery configured to receive the coil, and a raised central region corresponding to a center of the coil, wherein at least some of the at least three clips are in the raised central region.

7. The device of claim 5, wherein the at least one clip includes raised features separated by a distance corresponding to a diameter of the proximal lead end to receive the proximal lead end between the raised features.

8. The device of claim 7, wherein the proximal lead end includes an insulator coating that is compressed when the proximal lead end is inserted between the raised features.

9. The device of claim 1, further comprising a battery liner adjacent to at least a portion of a periphery of the battery, the battery liner including at least one spring configured to press against the periphery of the battery to resist battery movement.

10. The device of claim 9, wherein the battery liner is configured to be positioned along at least a width edge of the battery and a length edge of the battery, wherein the at least one spring includes one or more springs along the width edge of the battery to constrain battery movement in the length direction and one or more springs along the length edge of the battery to constrain the battery movement in the width direction.

11. The device of claim 9, wherein the at least one spring includes an arch spring.

12. The device of claim 1, further comprising a strain relief at a housing-lead interface where the at least one lead enters the housing.

13. The device of claim 12, wherein the strain relief includes side webs on each side of the at least one lead, each of the side webs extending from a first position that is near the coil frame and away from the leads to a second position that is near the leads and away from the coil frame.

14. The device of claim 13, further comprising a suture hole formed in at least one of the side webs.

15. The device of claim 13, further comprising a suture hole formed in each of the side webs.

16. The device of claim 1, wherein the device includes a bottom surface corresponding to a contour of a skull.

17. The device of claim 16, wherein the housing of the device includes;
a base mold having a first portion with a first planar bottom surface and a second portion with a second planar bottom surface, the first and second planar bottom surfaces forming an angle corresponding to a skull contour such that the bottom surfaces for both the first and second portions are approximately tangent to a skull surface;
a coil frame for holding a coil on the first portion of the base mold; and
a can assembly for housing the stimulator circuitry on the second portion of the base mold.

18. The device of claim 17, wherein the coil frame has an interface with the can assembly, each of the coil frame near the interface and the can assembly near the interface having features for enhancing an ability of epoxy to fix the coil frame to the can assembly, wherein the epoxy is provided over the interface and hardens to fix the coil frame to the can assembly.

19. The device of claim 18, wherein the features for enhancing the ability of the epoxy to fix the coil frame to the can assembly includes apertures in each of the coil frame and the can assembly, and wherein the epoxy at least partially fills the apertures.

20. The device of claim 17, further comprising a flexible tail, the flexible tail and the coil frame being on opposing sides of the can assembly, the base mold having a third portion with a third planar bottom portion, the flexible tail being formed on the third portion, the third portion having a bottom surface forming an angle with the bottom surface of the second portion such that the bottom surface of the third portion is approximately tangent to the skull surface.
